(19) United States
(12) Patent Application Publication
(10) Pub. No.: US 2016/0235560 A1
(43) Pub. Date: Aug. 18, 2016

(54) VARIABLE ELASTIC MODULUS CUSHION DISPOSED WITHIN A DISTAL CUP OF A PROSTHETIC SOCKET

(71) Applicant: LIM Innovations, Inc., San Francisco, CA (US)

(72) Inventors: Juan Jacobo Cespedes, San Francisco, CA (US); Jesse Robert Williams, San Francisco, CA (US); Garrett Ray Hurley, San Francisco, CA (US)

(21) Appl. No.: 15/047,131

(22) Filed: Feb. 18, 2016

Related U.S. Application Data
(60) Provisional application No. 62/117,501, filed on Feb. 18, 2015.
(60) Provisional application No. 62/128,218, filed on Mar. 4, 2015.
(60) Provisional application No. 62/163,717, filed on May 19, 2015.

(51) Int. Cl. A61F 2/78; A61F 2/80

(52) U.S. Cl. CPC A61F 2/7812; A61F 2/80; A61F 2002/785; B33Y 10/00

(57) ABSTRACT

A cushion for a distal cup of a prosthetic socket has a total volume that includes a solid thermoplastic composition volume and a void volume. A ratio of the solid composition relative volume to the total volume yields a relative thermoplastic elastomer fill volume that is correlated with an elastic modulus of the cushion. The relative thermoplastic elastomer fill volume can vary regionally within the cushion in a customized manner, and accordingly, so can the elastic modulus and the durometer. Varying the elastic modulus or durometer regionally within a cushion, such as by 3D printing the cushion, provides therapeutic and prophylactic benefits in the application of the cushion to articles that interface with the body, such as a cushion disposed in the distal portion of a prosthetic socket for a residual limb of an amputee.

### CROSS-REFERENCE TO RELATED APPLICATIONS

This patent application claims the priority and benefit of U.S. Provisional Application Nos. 62/117,501, filed Feb. 18, 2015, entitled “Strap-Based Mechanism for Suspending a Prosthetic Device on a Residual Limb,” and 62/128,218, filed Mar. 4, 2015, entitled “Modular Prosthetic Socket,” and 62/163,717, filed May 19, 2015, entitled “Prosthetic Socket Distal Cup with a Strap Lanyard Suspension Mechanism and a Variable Elastic Modulus Cushion.” All of the above-referenced applications are herein incorporated in their entireties by reference.

This patent application is further related to the following U.S. Patent Applications: application Ser. No. 13/675,761, entitled “Modular prosthetic sockets and methods for making same,” filed Nov. 13, 2012; application Ser. No. 14/213,788, entitled “Modular prosthetic sockets and methods for making and using same,” filed Mar. 14, 2014; and application Ser. No. 62/007,742, entitled “Apparatus and method for transferring a digital profile of a residual limb to a prosthetic socket strut,” filed Jun. 4, 2014. The above-referenced patent applications are hereby incorporated by reference in their entireties into the present patent application.

### INCORPORATION BY REFERENCE

All publications and patent applications identified in this specification are herein incorporated by reference to the same extent as if each such individual publication or patent application were specifically and individually indicated to be so incorporated by reference.

### TECHNICAL FIELD

The present application relates to a prosthetic socket system for a residual limb of an amputee. More specifically, the application relates to a suspension mechanism that retains the socket on the residual limb of the patient.

### BACKGROUND

The distal portion of a prosthetic socket has several particular functional responsibilities, including participating in suspension of the prosthetic socket on the residual limb and absorbing force that transmitted through the distal end of the residual limb in a manner that is as kind as possible to that distal end.

It is not enough that a prosthetic socket simply fit, however well, on the residual limb of a patient; it must also resist distal slippage, up and down pistoning when the user is walking, and rotation. In the prosthetic arts, resisting these forms of instability on the residual limb is referred to as “suspending” the socket. Suspension devices for prosthetic sockets that make use of a sock-like liner garment that fits over at least the distal portion of a residual limb are well known. These liners are typically formed from an air-impermeable elastomer material, such as silicone, and are configured to allow radial distension while being resistant to axial distension. Liners fit closely over, and conform to the shape of, the limb, particularly at their proximal end. The consistency of the silicone elastomer is friendly to the skin of the residual limb, and although it can easily be manually peeled away, it readily forms a substantially hermetic seal that effectively isolates the distal portion of the residual limb within the confines of the liner.

In addition to attributes of prosthetic socket liners that contribute to the security of the socket on the limb, solutions have been developed that mechanically secure or stabilize the liner within the socket. Suspension, thus, is typically achieved by a layered approach, with various mechanisms working together. Suspension is inherently difficult, however, and the residual limb needs to be treated gently, since damaging the skin on the residual limb is completely intolerable. Also, the residual limb generally does not provide points of mechanical advantage that could contribute to suspension. Accordingly, suspension solutions continue to be sought, particularly solutions that are not only effective, but simple in terms of mechanism, low profile design, ease in operation, and ease in donning and removal.

It is generally advantageous for a prosthetic socket to distribute the force associated with body weight and stride impact away from the distal end of the residual limb, because the distal end is sensitive, and lacks the natural force absorbing structure provided by intact limbs with a condyle. Nevertheless, at least some amount of body weight and impact force is transmitted from the distal end of the residual limb to a pad or cushion disposed within the distal base of the socket. Optimizing the configuration and composition of a distal cushion so that force conveyed through the distal end of the residual limb is absorbed in as benign a manner as possible would be a welcome development in the prosthetic arts.

### SUMMARY OF THE INVENTION

Embodiments of the invention are directed to a cushion within the distal portion of a prosthetic socket for use in hosting a residual limb of an amputee patient. Embodiments of such a distal cup include a proximal disk; a distal disk; and a cushion incorporated into the proximal disk that is custom manufactured specifically for the patient. Embodiments of the cushion include (1) a total volume of the cushion, (2) a volume of solid thermoplastic elastomer within the total cushion volume, and (3) a void volume within the total cushion volume. Based on these values, a relative fill volume can be defined as the ratio of (2) the total volume of solid thermoplastic elastomer vs. (1) the total volume of the cushion.

Embodiments of this cushion further include a first subset volume having a first relative thermoplastic elastomer fill volume, as defined by the ratio of solid thermoplastic elastomer volume within the first subset volume to the total volume of the first subset volume, and a second subset volume having a second relative thermoplastic elastomer fill volume, as defined by the ratio of solid thermoplastic elastomer volume within the second subset volume to the total volume of the second subset volume. In such embodiments, the first and second relative thermoplastic elastomer fill volumes are different from one another, and the first and second relative thermoplastic elastomer fill volumes are selected on the basis, at least in part, on at least one physical characteristic of the patient.

In some embodiments, the proximal disk and the distal disk each include a central well configured to allow passage of a strap lanyard of the prosthetic socket therethrough. In some embodiments, the proximal disk and the cushion are formed as a single monolithic structure.

In typical embodiments, the cushion is a 3D printed article, and the first and second relative thermoplastic elastomer fill volumes within the cushion (and differing from each other) are formed via a 3D printing process.

In some embodiments, the durometer of the cushion is selected for the patient by selecting the first and second relative thermoplastic elastomer fill volumes. And in some embodiments, the modulus of elasticity of the cushion is selected for the patient by selecting the first and second relative thermoplastic elastomer fill volumes.

In some embodiments of the cushion, beyond the first and second subset volumes, the cushion may further include a third subset volume within the total cushion volume, the third subset volume having a third relative thermoplastic elastomer fill volume as defined by a ratio of a solid thermoplastic elastomer volume within the third subset volume to the total volume of the third subset volume. The first, second and third relative thermoplastic elastomer fill volumes are different from one another. Beyond the third subset volume within the cushion, some embodiments may include a fourth subset volume within the total cushion volume, the fourth subset volume having a fourth relative thermoplastic elastomer fill volume as defined by a ratio of the solid thermoplastic elastomer volume within the fourth subset volume to the total volume of the fourth subset volume. The first, second, third, and fourth relative thermoplastic elastomer fill volumes are all different from one another.

In some embodiments of the cushion, the cushion has a spatial center and x, y, and z axes for reference; and any of the first, second, and third, and further multiple relative thermoplastic elastomer fill volumes may form a relative fill volume gradient along one or more of the x, y, and z axes.

In some embodiments of the cushion, the cushion has a spatial center surrounded by layers, such as ring-shaped or concentric spherical layers, emanating outward from the spatial center toward an external boundary of the cushion, and the first, second, and third, and further multiple relative thermoplastic elastomer fill volumes may form a relative fill volume gradient on a line from the center through the ring-shaped layers.

In some embodiments, the thermoplastic elastomer composition of the cushion includes multiple structures selected from the group consisting of spheroidal structures, ovoid structures, walled structures, filamentous structures and irregularly-shaped structures.

In some embodiments, the thermoplastic elastomer composition of the cushion includes one or more structures, each structure having one or more vector orientations; A modulus of elasticity of the cushion may vary through the cushion in accordance with the one or more vector orientations of the structures.

In some embodiments, the 3D printing process by which a cushion if formed is controlled by a control file comprising data providing an external boundary profile of the cushion as well as a map of the internal volume of the cushion. A parameter within the map of the internal volume includes specification of the levels of relative thermoplastic elastomer fill density. Such a map may be derived, at least in part, from a digital profile of the patient's residual limb.

In some embodiments, the at least one physical characteristic of the patient includes a digital profile of the patient's residual limb, and the external boundary profile of the cushion may be derived, at least in part, from the digital profile of the patient's residual limb.

In some embodiments, the control file includes data that prescribe a range of predetermined sizing options for the cushion. And in some embodiments, the control file includes data that prescribe a variety of predetermined shape options for the cushion. These data may be derived, at least in part, from the digital profile of the patient's residual limb.

In some embodiments, the at least one physical characteristic of the patient is selected from the group consisting of a weight of the patient, a height of the patient, a dimension of the residual limb, a digital profile of the residual limb, a status of an opposite limb of the patient, and a health condition of the patient.

Embodiments of the invention further include methods of making a distal cup of a prosthetic socket for a residual limb of a patient, such as the distal cup as summarized above. Embodiments of the method include fabricating a distal disk of the distal cup and fabricating a proximal disk of the distal cup (wherein the proximal disk includes a cushion custom manufactured specifically for the patient) and coupling the distal disk to the proximal disk to form the distal cup. Embodiments of the method may further include using the distal cup for the assembly of a complete prosthetic socket.

Embodiments of a cushion fabricated by this method include (1) a total volume of the cushion, (2) a volume of solid thermoplastic elastomer within the total cushion volume, and (3) a void volume within the total cushion volume. Based on these values, a relative fill volume can be defined as the ratio of the volume of solid elastomer to the total volume of the cushion.

Cushion embodiments further include a first subset volume having a first relative thermoplastic elastomer fill volume, and having a second subset volume having a second relative thermoplastic elastomer fill volume. In such embodiments, the first and second relative thermoplastic fill volumes are designated based, at least in part, on at least one physical characteristic of the patient, such as, by way of example, a digital profile of at least a portion of the residual limb. In such embodiments, the first and second relative thermoplastic elastomer fill volumes may be different from one another, and the first and second relative thermoplastic elastomer fill volumes are selected based at least in part on at least one physical characteristic of the patient.

In some embodiments of the method, at least the distal disk of the cushion is fabricated via a 3D printing method that includes providing instructions to a 3D printer via a control file, and printing the cushion with the 3D printer in accordance with the instructions. In such embodiments, the control file includes a map of an external boundary of the cushion and a map of a total internal volume of the cushion, the internal volume including the first and second relative thermoplastic elastomer fill volumes.

In some embodiments of the method, the instructions specify that the thermoplastic elastomer composition include multiple structures selected from the group consisting of spheroidal structures, ovoid structures, walled structures, filamentous structures, and irregularly-shaped structures.

In some embodiments of the method, the instructions within the control file specify that the solid thermoplastic composition volume include one or more structures, each structure providing one or more strength vector orientations, and a modulus of elasticity of the cushion that varies through the cushion in accordance with the one or more strength vector orientations of the structures.

In some embodiments of the method, the instructions within the control file specify that the cushion comprise a spatial center and x, y, and z axes, and wherein the first and second relative thermoplastic elastomer fill volumes together form a relative fill volume gradient along one or more of the x, y, and z axes.

In some embodiments of the method, the instructions within the control file specify the cushion provide a spatial center surrounded by ring-shaped layers emanating outward from the spatial center toward an external boundary, and the first and second relative thermoplastic elastomer fill volumes together form a fill volume gradient on a line from the center through the ring-shaped layers.

In some embodiments of the method, the instructions within the control file specify that the first relative thermoplastic elastomer fill volume include an insular volume and the second relative thermoplastic elastomer fill volume comprises a region surrounding the insular volume.

### BRIEF DESCRIPTION OF THE DRAWINGS

FIGS. 1A-1E show top, left side, front side, right side, and bottom views, respectively, of a prosthetic socket distal cup, having a strap-based lanyard suspension mechanism and a variable elastic modulus cushion, according to one embodiment.

FIGS. 2A-2B show a top perspective view and a bottom perspective view, respectively, of an embodiment of a prosthetic socket distal cup, in which the angle of the external surface of the distal cup is variable.

FIGS. 3A-3B show a top perspective view and a bottom perspective view, respectively, of an embodiment of a prosthetic socket distal cup, showing in particular, a partially rotatable aspect thereof.

FIGS. 4A-4C show a cross sectional upper perspective view, a facing side view, and a cross-sectional side view, respectively, of an embodiment of a prosthetic socket distal cup. The distal cup includes a proximal disk that includes a 3D-printed elastomer in a pattern.

FIGS. 5A-5D show a top view, top perspective view, side cross sectional view, and top perspective cross sectional view, respectively, of a distal portion of an embodiment of a prosthetic socket distal cup.

FIGS. 6A-6C show a top perspective cross sectional view, a facing side view, and a cross sectional side view of a proximal portion of an embodiment of a prosthetic socket distal cup, showing, in particular, a view of variable elastic modulus cushion included in the proximal portion of the distal cup.

FIGS. 7A-7B show a top perspective cross sectional view of two alternative embodiments of the variable elastic modulus cushion within the proximal portion of the distal cup. The distal cup includes a proximal disk that includes a 3D-printed elastomer in a pattern that can vary in relative density from region to region.

FIGS. 8A-8C show a top perspective cross sectional view of alternative embodiments of the variable elastic modulus cushion within the proximal portion of the distal cup, wherein the internal structure of the variable elastic modulus cushion is the same, but the thickness of the surrounding skin of the variable elastic modulus cushion varies.

FIG. 9 shows a cross sectional side view of an embodiment of a prosthetic socket distal cup that details aspects of the suspension mechanism, including a lanyard strap threaded through a strap channel and the internal disposed distal end of the strap with an attachment feature for attaching to the distal end of a prosthetic socket liner.

FIG. 10 shows a side view of an embodiment of a prosthetic socket with a distal cup having a strap lanyard suspension mechanism, showing, in particular, the proximal portion of the strap as it is attached to a strut sleeve arranged over a socket strut.

FIG. 11 shows a side view of an embodiment of a prosthetic socket with a distal cup having a strap lanyard suspension mechanism, as in FIG. 10, further showing a proximal brim element arranged over the proximal portion of the socket, thus obscuring the site of attachment to the strut sleeve.

FIGS. 12A-12B show a side view and a detailed view, respectively, of an embodiment of a prosthetic socket with a prosthetic socket cup with suspension features, showing, in particular, a side view of the proximal portion of the strap as it connects to a connecting element attached to a strut sleeve.

FIG. 13 shows a side view of an embodiment of a prosthetic socket with a prosthetic socket cup with suspension features that is similar to FIG. 12A, except that a prosthetic socket liner is now shown within the socket, the attachment of the distal end of the suspension strap and its connection to the distal end of the liner being shown transparently through a strut. The prosthetic socket liner is not fully pulled down into the distal end of the prosthetic socket.

FIG. 14A shows a side view of an embodiment of a prosthetic socket with a prosthetic socket cup with suspension features that is similar to FIG. 13, except that the prosthetic liner is now fully pulled into the distal base of the socket, which is shown in these cross sectional views.

FIG. 14B shows a detail view of a distal portion of the prosthetic socket embodiment shown in FIG. 14A.

FIG. 15 shows a shin guard as an example of an article that includes a cushion having a 3D-printed elastomer matrix.

FIG. 16 shows a helmet as an example of an article that includes a cushion having a 3D-printed elastomer matrix that includes regions that vary in their elastomeric material density, and accordingly, varying in elastic modulus.

FIG. 17 shows a boot liner as an example of an article that includes a cushion having a 3D-printed elastomer matrix.

### DETAILED DESCRIPTION OF THE INVENTION

Aspects of the technology disclosed herein are directed toward improvements in and around the distal base portion of a prosthetic socket. In one aspect, the technology relates to mechanics of suspending a prosthetic socket on a residual limb. These improvements include a strap-lanyard suspension arrangement.

In another aspect, the technology relates to a cushion that engages and supports the distal portion of the residual limb. A variable elastic modulus cushion is positioned within proximal disk or shock absorbing pad of prosthetic socket distal cup. Embodiments of the cushion may be 3D printed, and they may include variable and customizable patterns of elasticity and durometer that can be directed to optimizing fit and therapeutic benefit. These patterns are a result of at least two variables: (a) the variation in thermoplastic elastomer fill density (solid volume/total volume) within regional subsets of the total volume, and (b) the structures formed by the interplay of solid and voids.

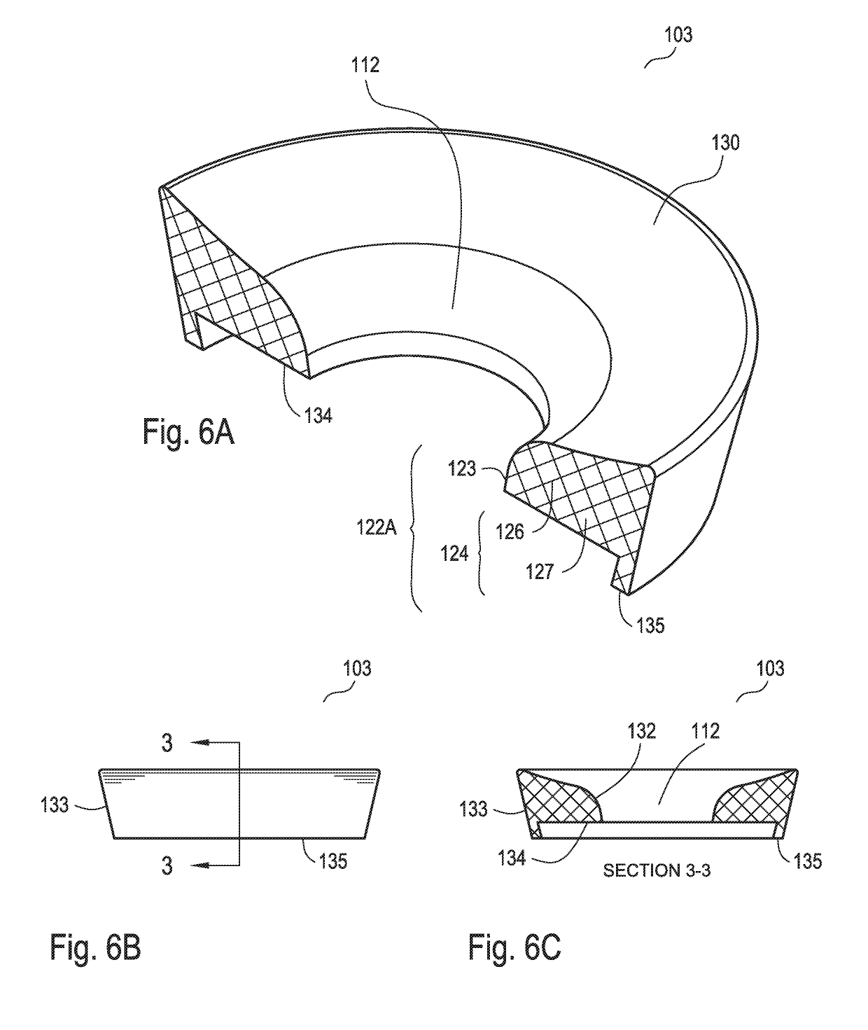

Aspects of a variable elastic modulus cushion 122 are shown in particular detail in FIGS. 6A-8C. Although embodiments of cushion 122 are shown and described in the context of the distal portion of a prosthetic socket, embodiments of the technology have broader applicability in other medical devices, wearable articles, and personal assistive devices.

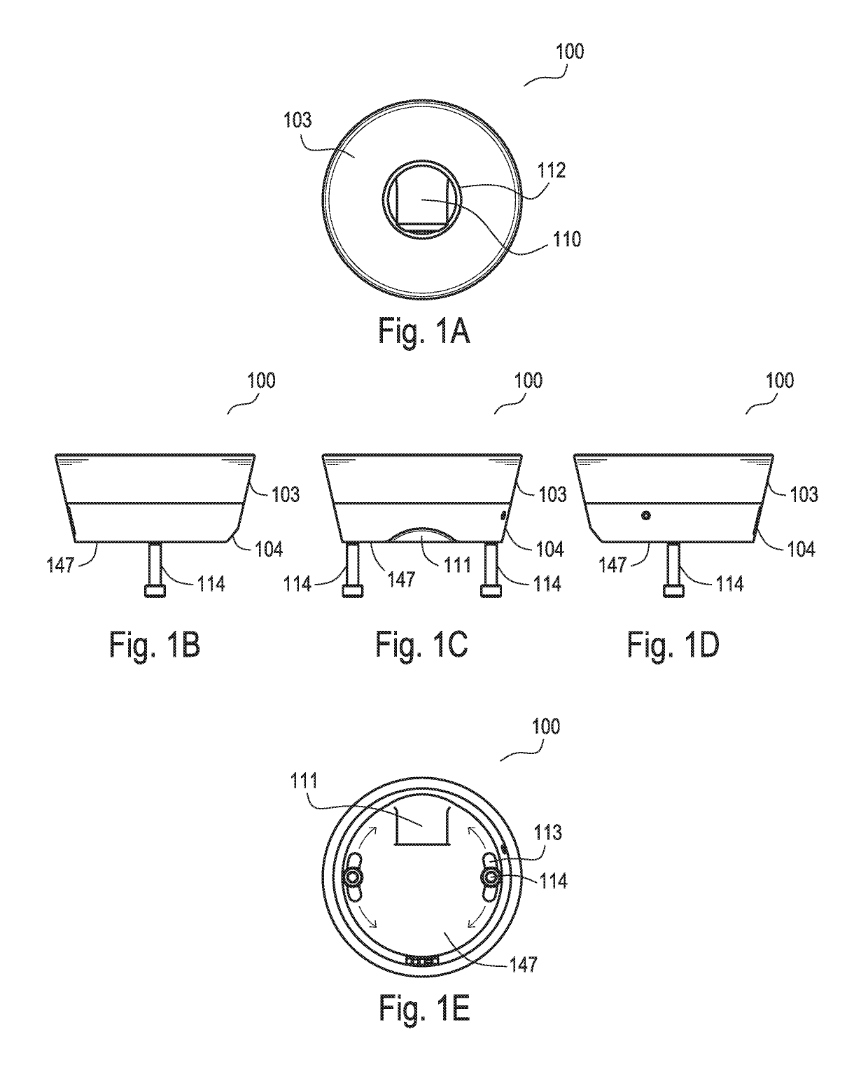

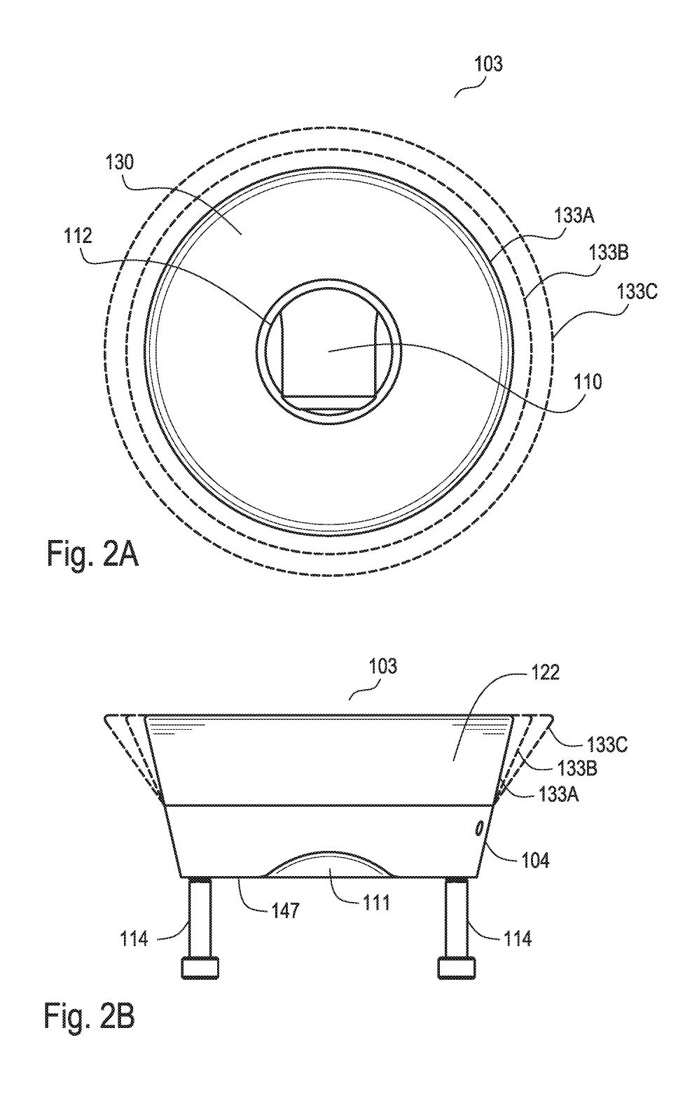

FIGS. 1A-1E show top, left side, front side, right side, and bottom views, respectively, of an embodiment of a prosthetic socket distal cup 100 having a strap-based lanyard suspension mechanism having a proximal disk 103 and distal disk 104, the interior of proximal disk 103 containing a variable elastic modulus cushion (not visible in FIGS. 1A-1E; see 122 in FIGS. 6A-8C). Distal cup 100, in this embodiment, includes proximal disk 103 and distal disk 104 that are distinct pieces, and the variable elastic modulus cushion is contained within proximal disk 103. In alternative embodiments, however, proximal and distal disks may be formed as one, integral disk, and/or a variable elastic modulus may be fully integral with the integral disk. As seen variously in FIGS. 2A-8B, proximal disk 103 has an upper or proximal surface 130, and distal disk 104 has an upper or proximal surface 140.

FIG. 1A provides a view of central well 112 that includes a through opening in both proximal disk 103 and distal disk 104. Below central well 112, a portion of an angled strap channel 110 can be seen. Distal disk 104 has a degree of rotatability that is enabled by an arrangement wherein fixation bolts 114 that are embedded in proximal disk 103 and project distally through kidney shaped bolt holes 113 in distal disk 104. When bolts are loose, distal disk 104 can rotate with respect to proximal disk 103; when the bolts are tightened, or nuts engaging the bolts are tightened, rotation is locked. This rotation capability (indicated by arrows) allows a wearer to rotate distal disk 104 so that strap channel exit 111 is positioned most conveniently for the wearer.

FIGS. 2A-2B show a top perspective view and a bottom perspective view, respectively, of an embodiment of a prosthetic socket distal cup 100, wherein the angle of the external surface of the distal cup is variable. The variation in external angle (indicated by 133A, 133B, and 133C) allows the distal cup 100 to be provided in varied circumferential or diagonal sizes.

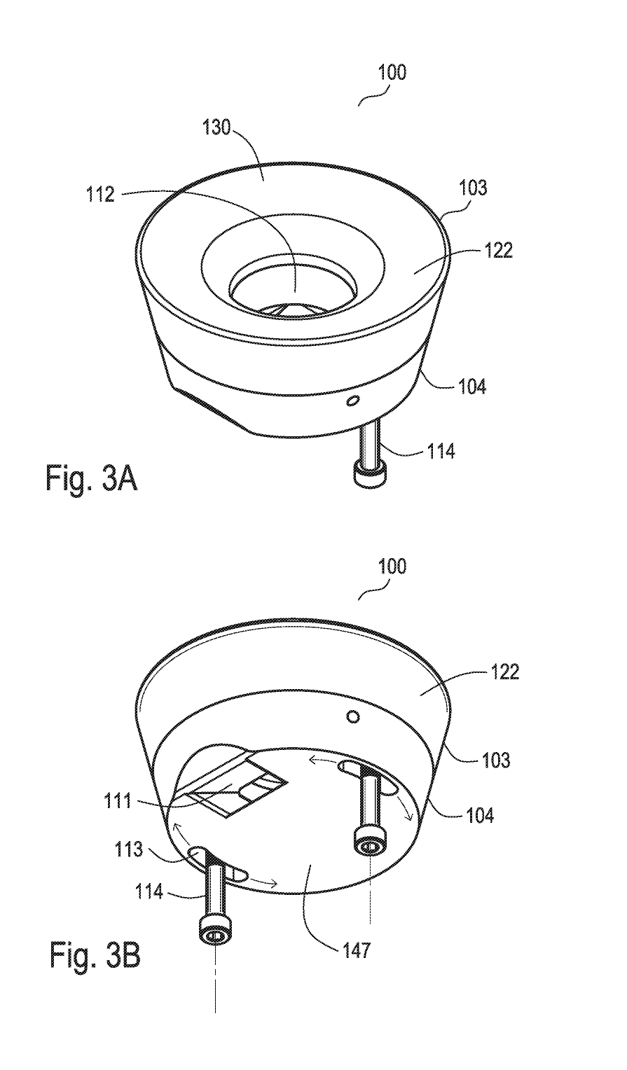

FIGS. 3A-3B show a top perspective view and a bottom perspective view, respectively, of an embodiment of a prosthetic socket distal cup 100, showing in particular, a partially rotatable aspect thereof. FIG. 3B, in particular, shows fixation bolts 114 emerging through kidney shaped bolt holes 113 (arrows indicate the rotational capability within the constraints of the bolt holes).

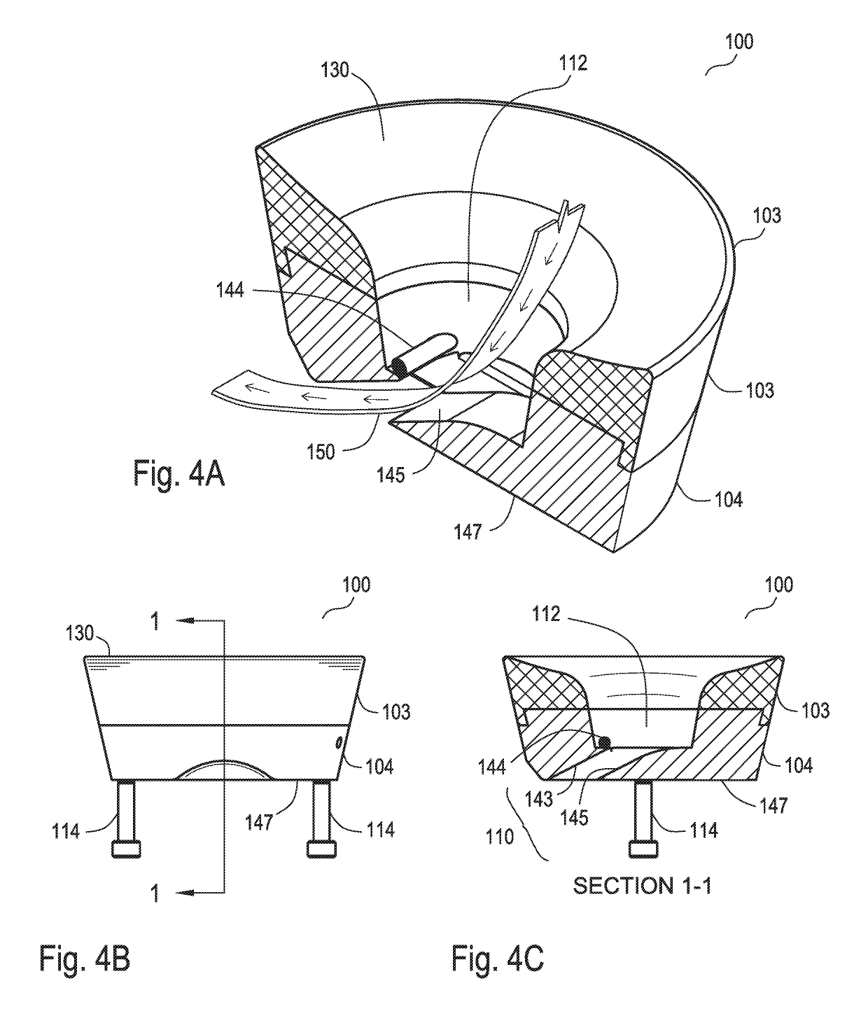

FIGS. 4A-4C show a cross sectional upper perspective view, a facing side view, and a cross-sectional side view, respectively, of an embodiment of a prosthetic socket distal cup 100. These figures provide illustrative views of strap 150 and its course through distal cup 100, more particularly through central well 112, and angled strap channel 142 through distal disk 103. Dowel guide 144 is positioned along the upper or proximal surface 143 of strap channel 142, and provides a smooth surface for strap 150 to be moved across when the strap is being tightened to secure a prosthetic socket liner in place.

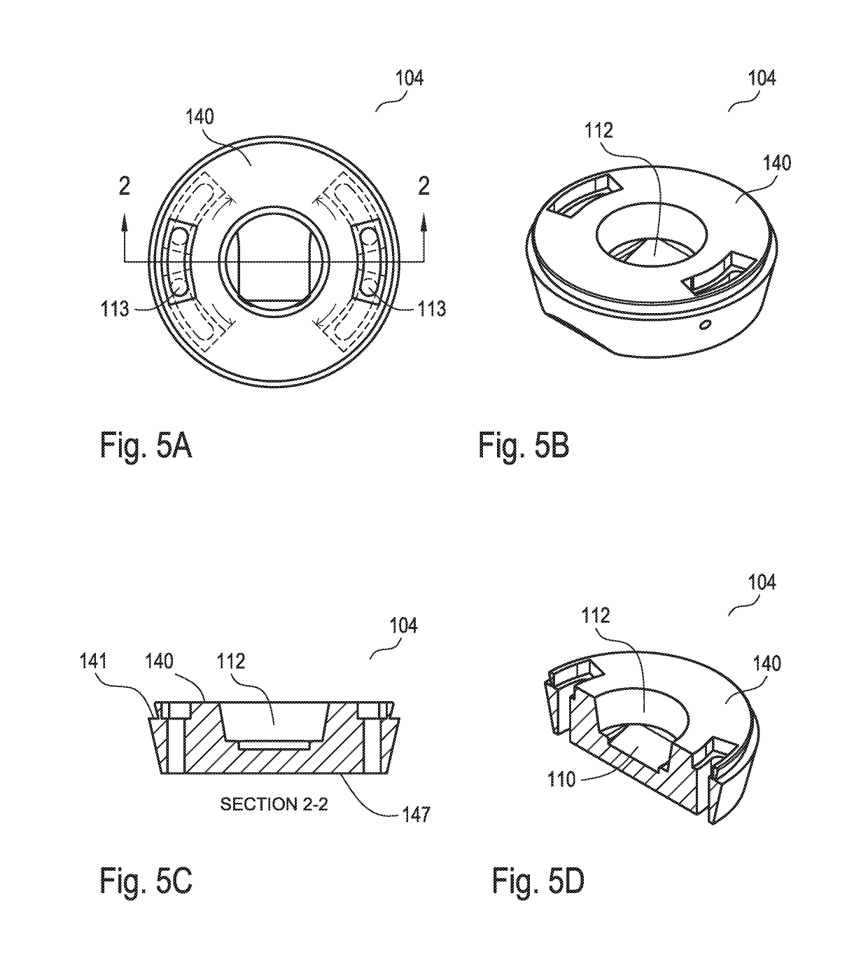

FIGS. 5A-5D show a top view, top perspective view, side cross sectional view, and top perspective cross sectional view of a distal disk 104 portion of an embodiment of a prosthetic socket distal cup 100.

FIGS. 6A-6C show a top perspective cross sectional view, a facing side view, and a cross sectional side view of a proximal portion of an embodiment of a prosthetic socket distal cup, showing, in particular, a view of variable elastic modulus cushion 122A included in the proximal disk 103 of the distal cup 100. The function of this cushion is to provide a friendly surface for the distal end of a wearer's residual limb to engage. Variable elastic modulus cushion 122 is formed by a 3D printing process, which can create patterns of variable material density.

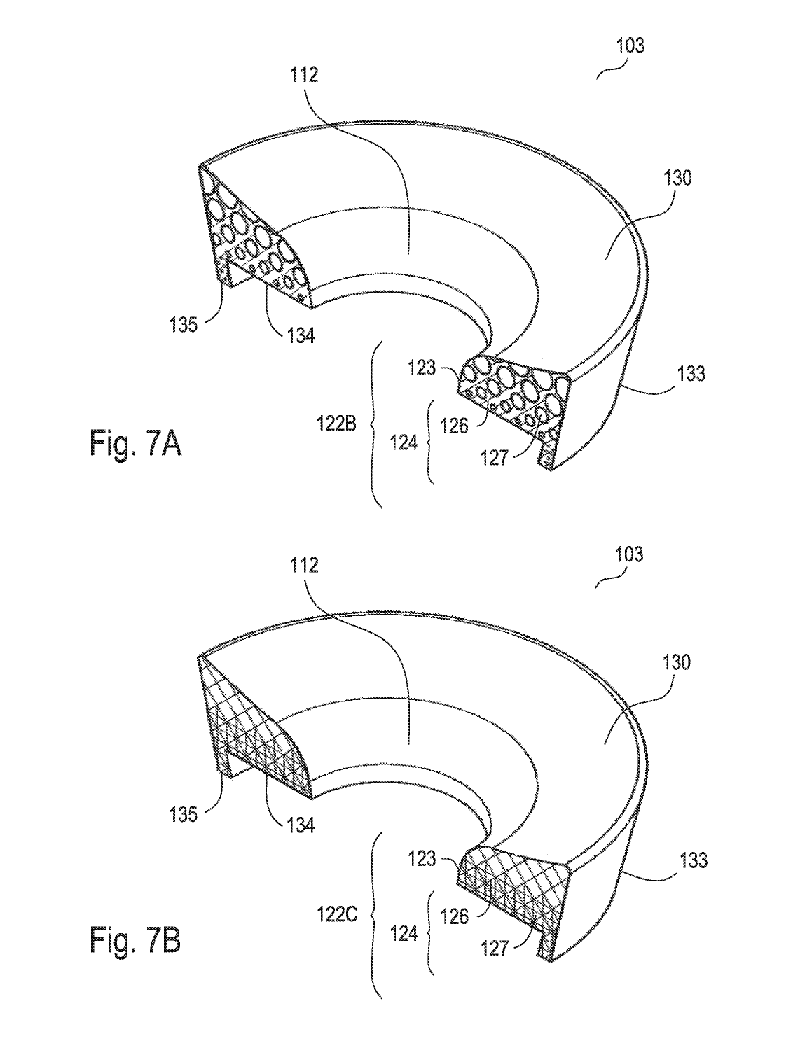

FIGS. 7A-7B show a top perspective cross sectional view of two alternative embodiments of the variable elastic modulus cushion 122 within the proximal portion of the distal cup 100. Whereas pattern shown in the padding 122A of proximal disk 103 of FIG. 6A shows a substantially uniform density throughout, the padding 122B (FIG. 7A) and 122C (FIG. 7B) differ. Padding 122B includes open cells 127 within a solid matrix 126. Open cells 127 are relatively large in the upper portion of padding 122B and relatively small in the lower portion. Thus, the overall material density is less at the top, and more at the bottom. By such an arrangement, the elasticity within cushion 122B varies, being of high elasticity at the top and low elasticity at the bottom.

Padding 122C (FIGS. 7B) includes filaments or sheets 128 that are arranged at various angles and at various spatial densities. In general, the density is relatively high in the upper portion of padding 122C and relatively low in the lower portion. Thus, the overall material density is less at the top, and more at the bottom. By such an arrangement, the elasticity within cushion 122B varies, being of high elasticity at the top and low elasticity at the bottom.

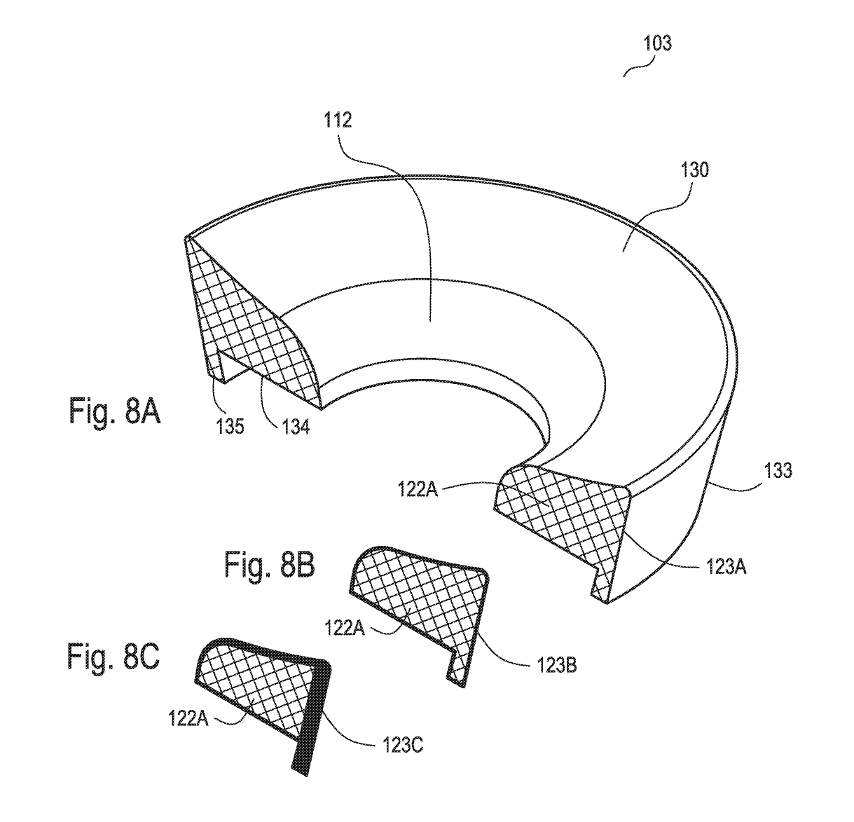

FIGS. 8A-8C show a top perspective cross sectional view of alternative embodiments of the variable modulus of elasticity cushion 122 within the proximal portion of the distal cup 100, wherein the internal structure of the variable modulus of elasticity cushion 122 is the same, but the thickness of the surrounding skin 123 of the variable modulus of elasticity cushion 122 varies. By way of example, skin 123A (FIG. 8A) is relatively thin, skin 123B (FIG. 8B) is of medium thickness, and skin 123C (FIG. 8C) is maximally thick. Skin portion 123 of a cushion 122 is fabricated by establishing a region of 3D printing in which the thermoplastic elastomer is laid down at a high density such that the relative fill volume is substantially 100%.

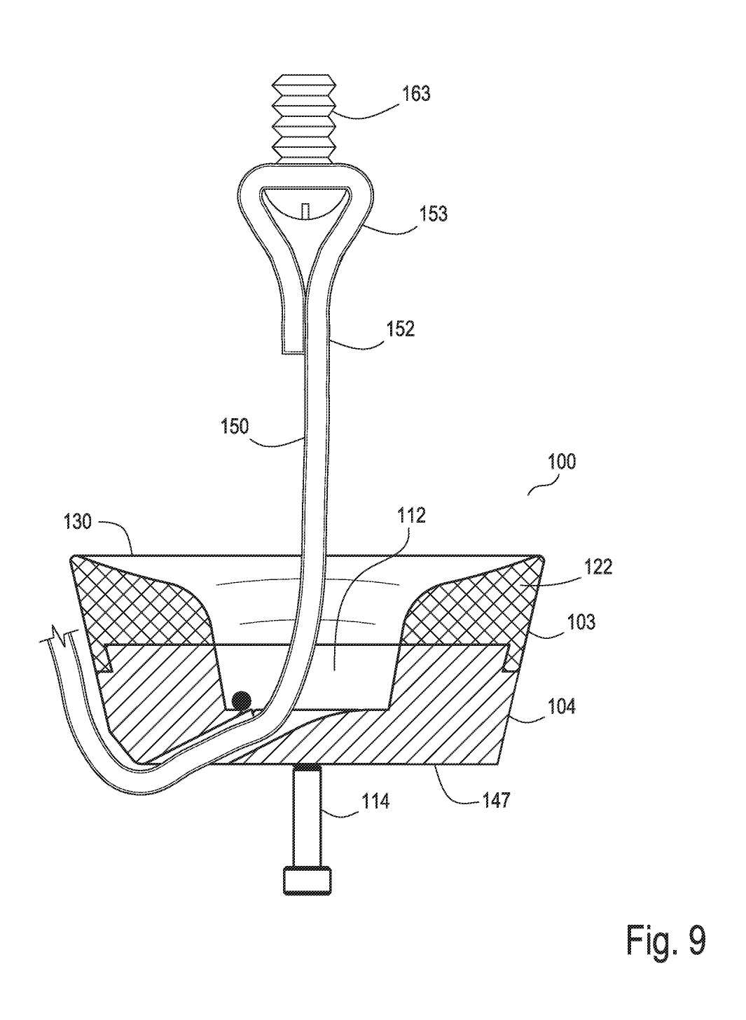

FIG. 9 shows a cross sectional side view of an embodiment of a prosthetic socket distal cup 100 that details aspects of the suspension mechanism, including a strap lanyard 150 threaded through a strap channel 142 and the internal distal end 152 of the strap 150 with an attachment feature 153 for attaching to an companion attachment feature 163 positioned at the distal end of a prosthetic socket liner (not shown here). The lower portion of FIG. 9 provides a view that is similar to that of FIG. 4A. Briefly, strap 150 courses through distal cup 100, more particularly through central well 112, and angled strap channel 142 within distal disk 103. Dowel guide 144 is positioned along the upper or proximal surface 143 of strap channel 142, and provides a smooth surface for strap 150 to be moved across when the strap is being tightened to secure a prosthetic socket liner in place.

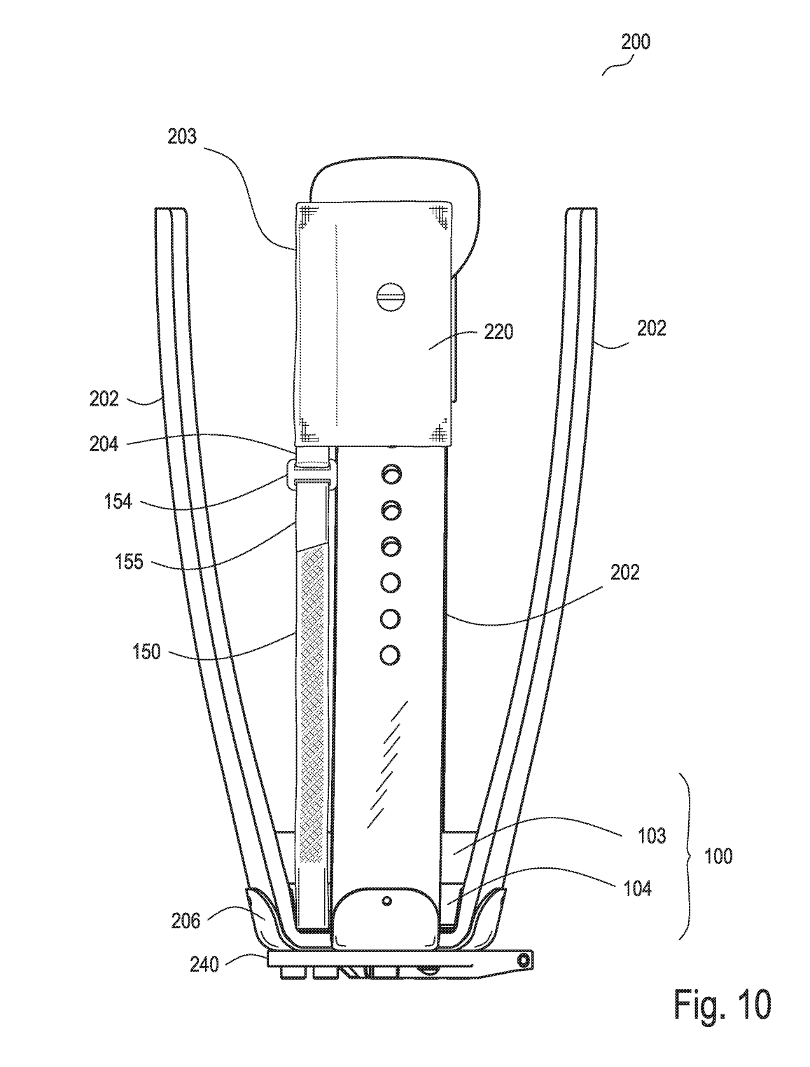

FIG. 10 shows a side view of an embodiment of a prosthetic socket 200 with a distal cup 100 having a strap lanyard suspension mechanism, showing, in particular, the proximal portion 155 of the strap 150 as it is attached to a strut sleeve 203 arranged over a socket strut 202 that is supporting a telescopically adjustable strut cap 220. Distal disk 104 and proximal disk 103 of distal cup 100 are seen tucked into the distal interior of prosthetic socket 200, as well as prosthetic socket distal base plate 240. An attachment element such as a buckle connects proximal end 155 of strap 150 to a mating element 204 that is attached to sleeve 203. Thus, proximal end 155 of strap 150 is ultimately connected to the structure of prosthetic socket 200.

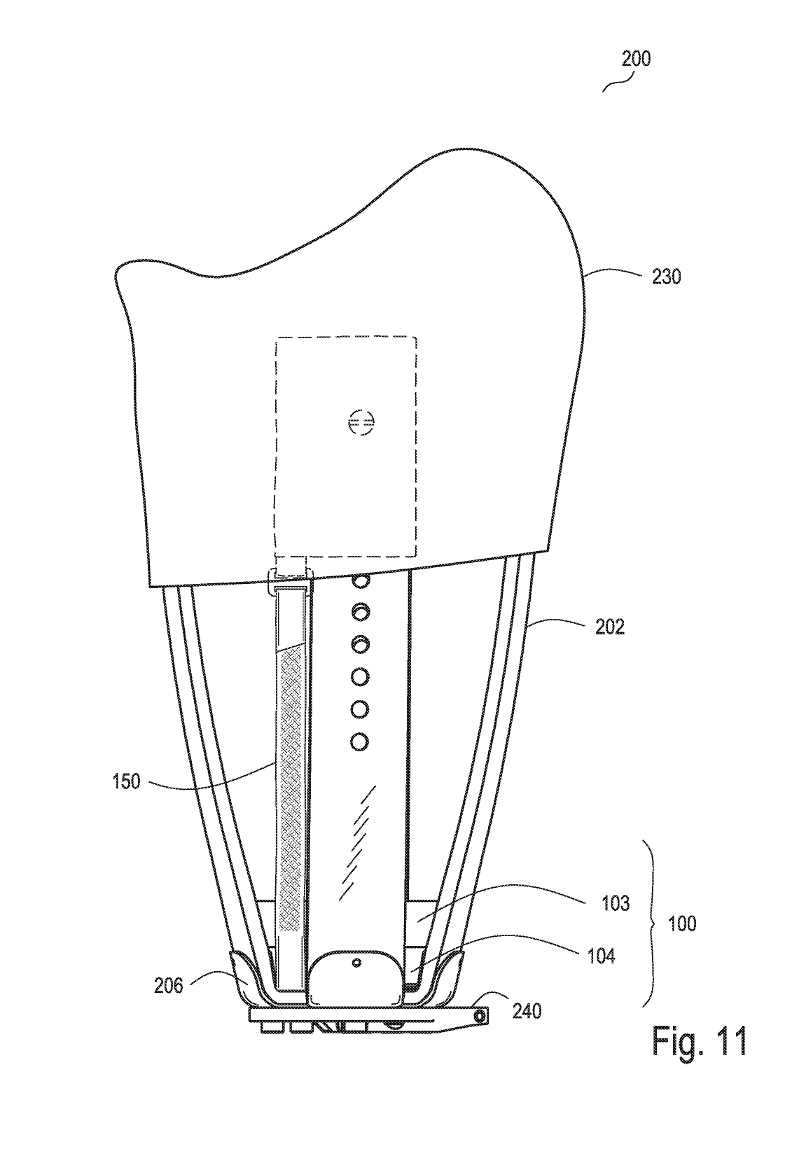

FIG. 11 shows a side view of an embodiment of a prosthetic socket 200 with a distal cup 100 having a strap lanyard suspension mechanism, as in FIG. 10, but further showing a proximal brim element 230 arranged over the proximal portion of the socket, thus obscuring the site of attachment to the strut sleeve.

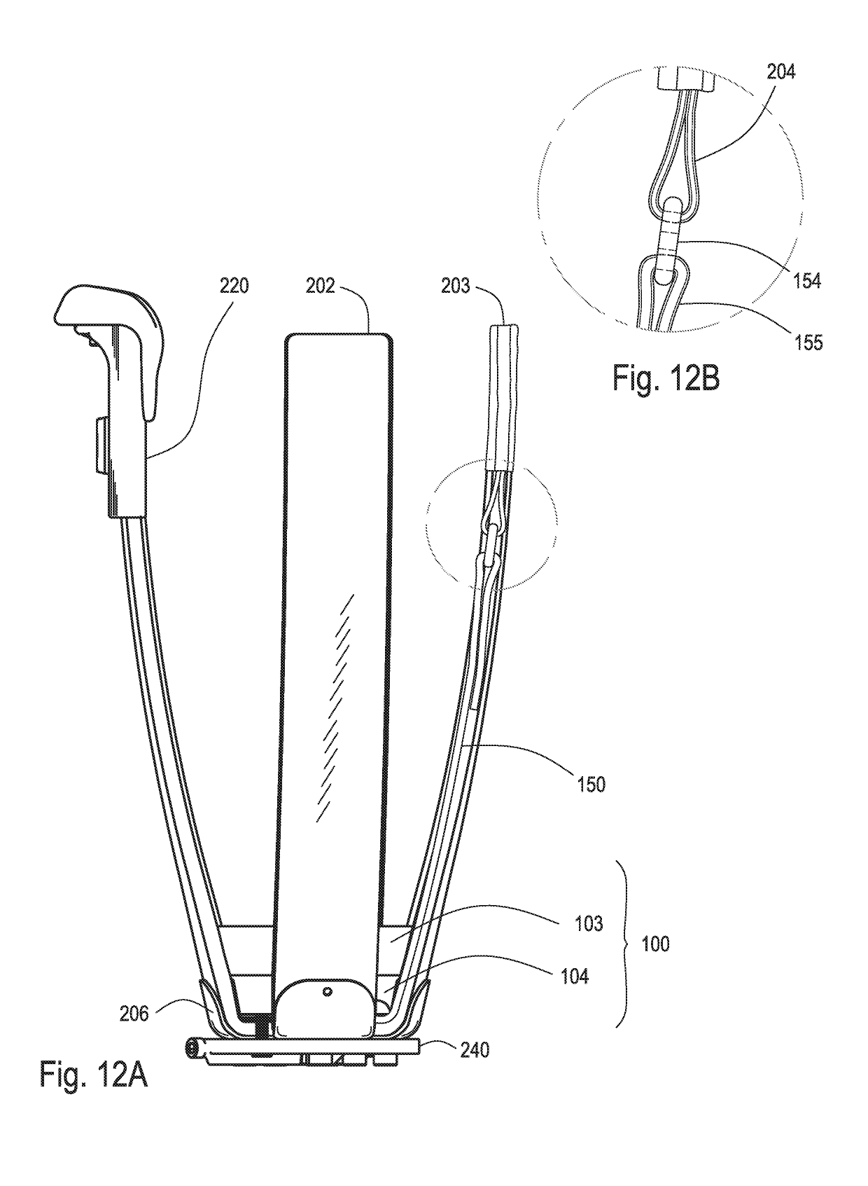

FIGS. 12A-12B show a side view and a detailed view, respectively, of an embodiment of a prosthetic socket 200 with a prosthetic socket cup 100 with suspension features, showing, in particular, a side view of the proximal portion of the strap 150 as it connects to a connecting element attached to a strut sleeve. FIG. 12B is a detailed view of the region of FIG. 12A where proximal end 155 of strap 150 is adjustably connected by way of attachment feature 154 to a mating piece 204 that is attached to strut sleeve 203.

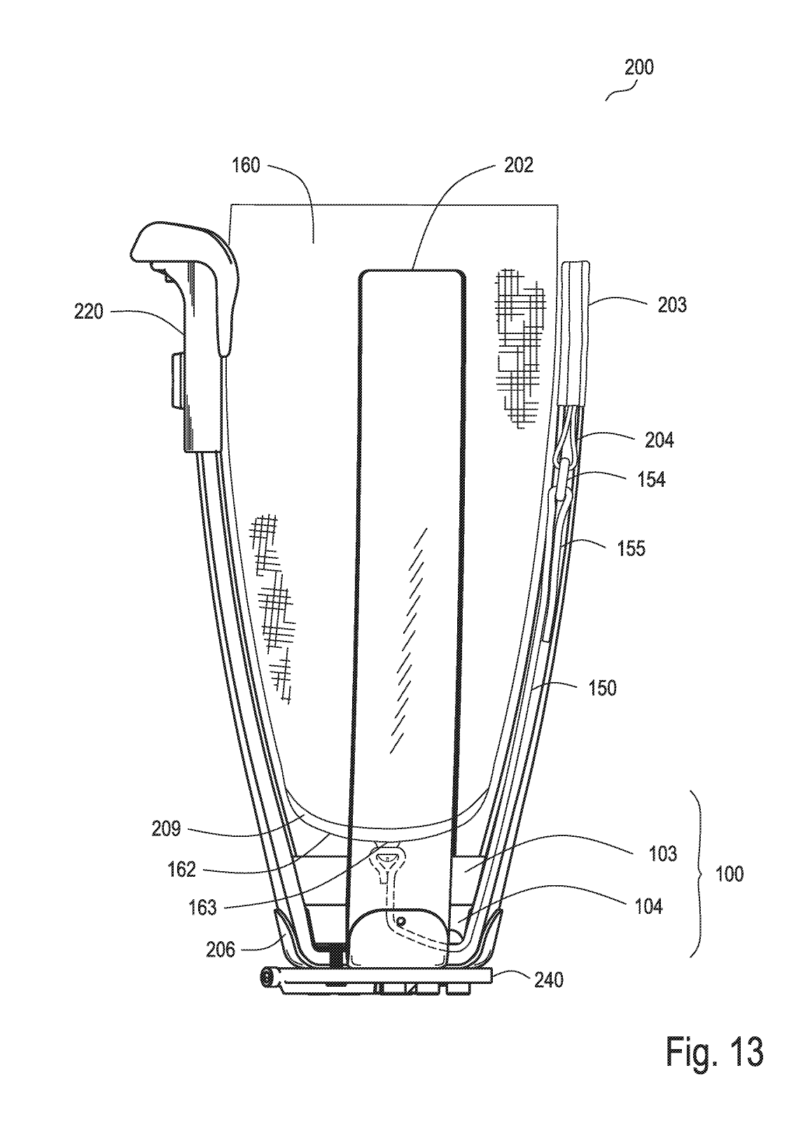

FIG. 13 shows a side view of an embodiment of a prosthetic socket 200 with a prosthetic socket cup 100 with suspension features that is similar to FIG. 12A except that a prosthetic socket liner 160 is now shown disposed within the socket, the attachment of the distal end 152 of the suspension strap 150 and its connection to the distal end 162 of the liner 160 being shown transparently through a strut. Prosthetic socket liner 160 is not fully pulled down into the distal end of the prosthetic socket 200.

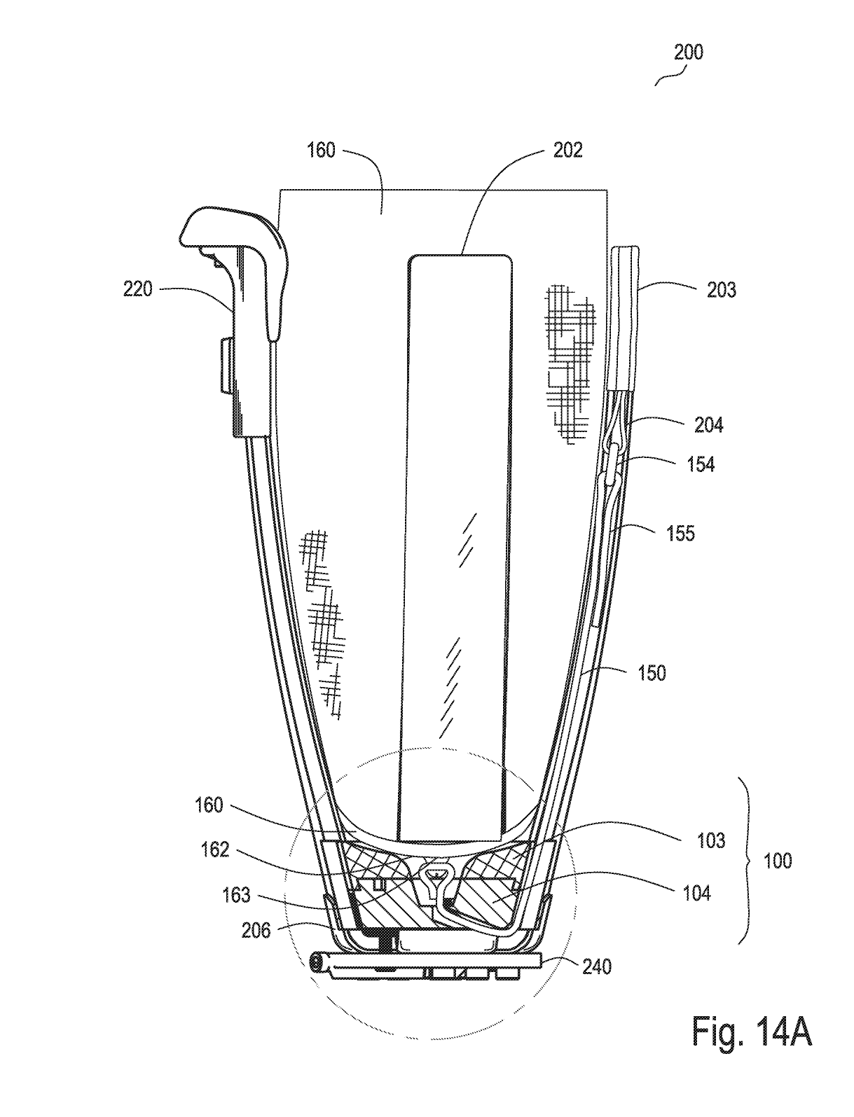

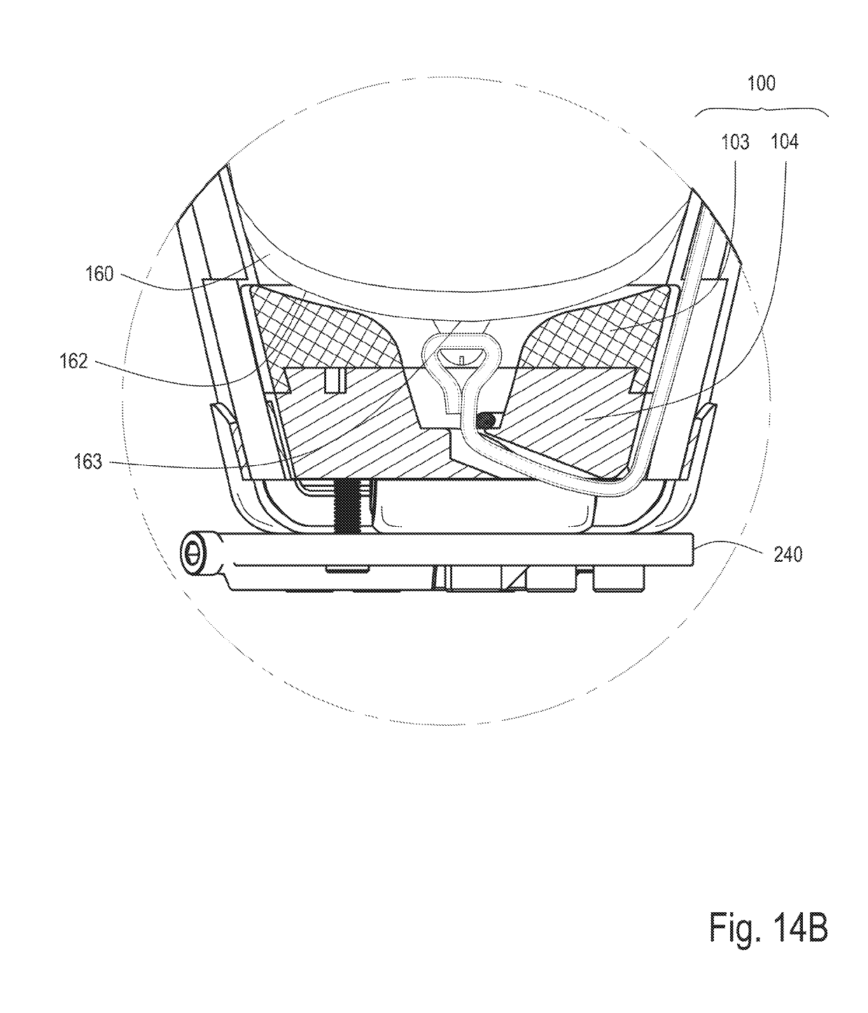

FIGS. 14A-14B show a side view an embodiment of a prosthetic socket 200 with a prosthetic socket cup 100 with suspension features that is similar to FIG. 13 except that the prosthetic liner is now fully pulled into the distal base of the socket, which is shown in cross section. FIG. 14B shows the cross sectional view of the prosthetic socket cup 100 in greater detail.

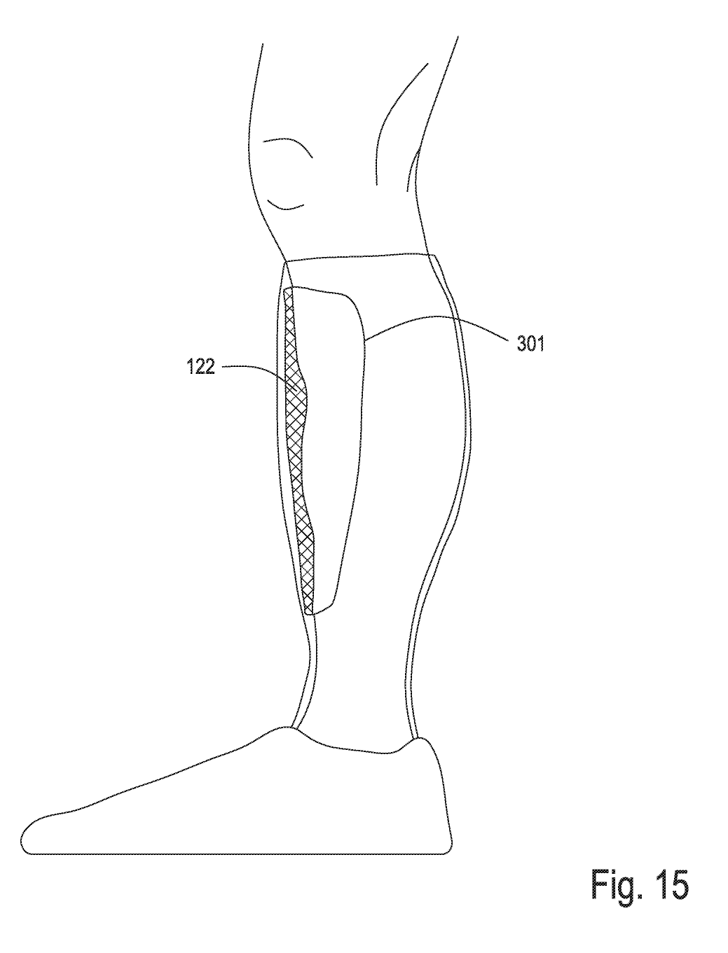

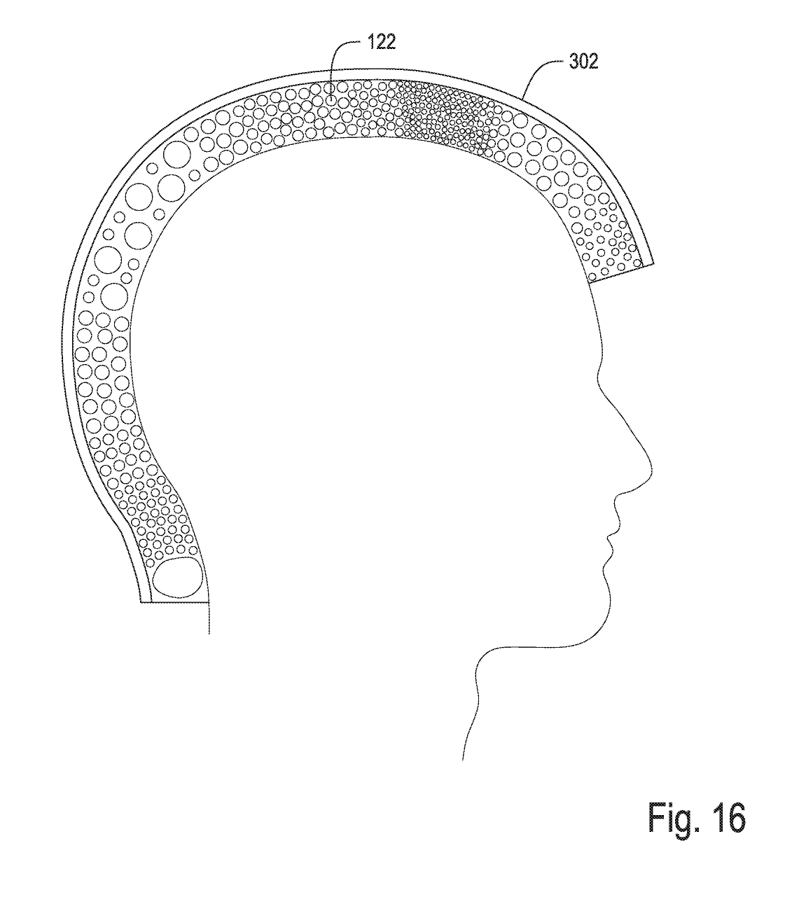

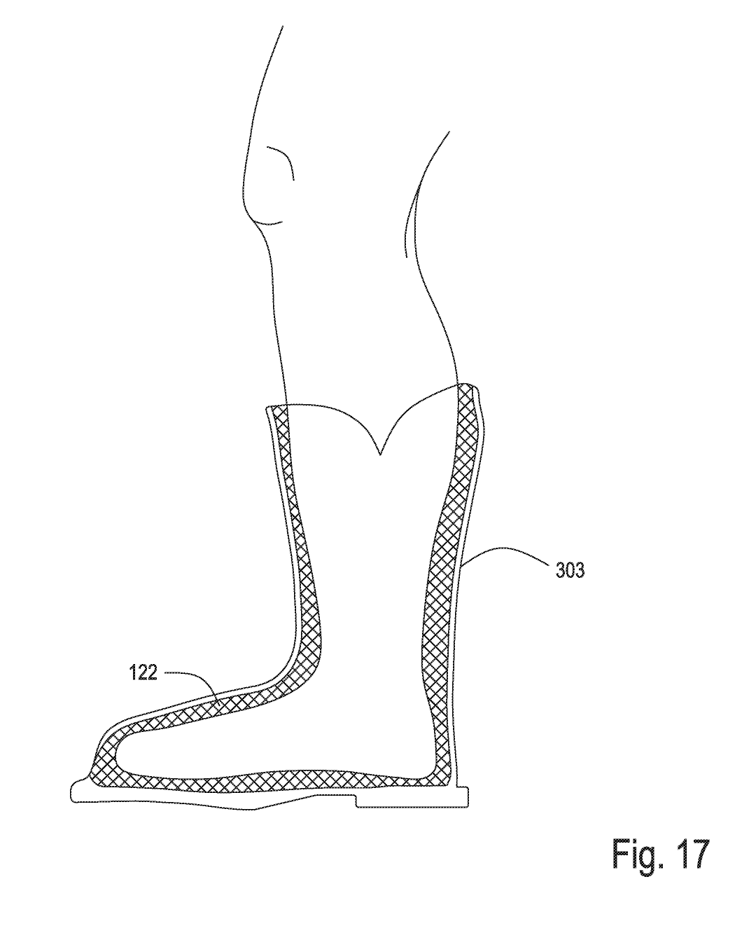

FIGS. 15-17 show examples of articles that incorporate cushion elements akin to those employed in the proximal disk 103 of FIGS. 4A, 6A, 7A-8C. FIG. 15 shows a shin guard 301 as an example of an article that includes a cushion having a 3D-printed elastomer matrix 122. FIG. 16 shows a helmet 302 as an example of an article that includes a cushion having a 3D-printed elastomer matrix 122 that includes regions that vary in their elastomeric material density, and accordingly, varying in elastic modulus. FIG. 17 shows a boot liner 303 as an example of an article that includes a cushion having a 3D-printed elastomer matrix 122.

As noted above, suspension of a prosthetic socket may be established by multiple mechanisms, each mechanism contributing to the totality of the stability of a prosthetic device on a residual limb. FIGS. 1-14B show various embodiments of strap type of lanyard mechanism that establishes a releasable mechanical connection between a prosthetic socket liner and the prosthetic socket.

Innovative aspects of the strap-based suspension mechanism disclosed herein include an angled (e.g., non-horizontal) strap channel 110 that has the functional advantage of providing a more effective distal pull on socket liner 160, pulling it securely into the base of a prosthetic socket 100. Distal end of 152 or strap 150 approaches its connection to the distal end of prosthetic socket liner 160 at an angle, and the distal end attachment feature 153 of the strap thus is configured to compensate, and direct the pull in a substantially distal direction. The external exit 111 of strap channel has a rotatable aspect that has the functional advantage of allowing the wearer a choice of angles for ease in manually pulling the strap. Finally, the proximal (exterior) end 155 of strap 150 attaches to an exterior site on prosthetic socket 200, more particularly to a buckle or attachment site 204 disposed on a strut sleeve 203. Attaching strap 150 to the socket component 200 advantageously stabilizes liner 160 within socket 200, and stabilizes the liner particularly against rotation within the socket. Another particularly innovative aspect of the strap-based suspension mechanism disclosed herein includes a variable elastic modulus cushion 122, which is described further below.

In some embodiments, a distal cup, rather than or in addition to a socket liner per se, may be the internal prosthetic element connected to an enclosing socket structure. The function of the releasable mechanical connection is to draw the prosthetic liner 160 downward (proximally) into the base of prosthetic socket 200 by way of a strap or cord 150 attached to the distal aspect 162 of the liner 160. When liner 160 is drawn into the socket 200 it is securely positioned such that pistoning movement (up and down) and rotational movement of the liner within the socket are both substantially prevented.

In typical embodiments of the disclosed technology, a prosthetic socket suspension mechanism includes a distal prosthetic socket cup 100 that includes a strap channel 110 with an exterior opening 111 into a central well 112 of the prosthetic socket cup 100. Notably, strap channel 110 is disposed at an upwardly directed angle (upward from the exterior opening and extending inward) toward the central well 112 of cup 100. The upwardly directed angle is typically within a range of about 10 to about 40 degrees from horizontal. Horizontal, in this context, refers to a plane orthogonal to the central longitudinal axis of the prosthetic socket.

In some embodiments, the distal cup 100 includes an upper (proximal) disk 103 and a lower (distal) disk 104, the aforementioned central well 112 being formed within both the distal and proximal disks. In some of these embodiments, the distal and proximal disks are rotatable and lockable with respect to each other within a range of about 10 degrees to about 25 degrees. The exterior opening 111 of strap channel 110 is generally disposed on the lateral aspect of the prosthetic socket where the patient can easily manipulate strap 150 to adjust the tension, lock the strap, and release the strap. This freedom of rotational movement allows the individual prosthetic socket user to position the strap at a location that is most comfortable and appropriate for the user.

When assembled, embodiments of the prosthetic socket suspension mechanism include a lanyard strap 150 disposed through the strap channel 110; strap 150 includes an exterior or proximal end 155, an interior or distal end 152, and a longitudinal axis. Exterior refers to residing exterior to the prosthetic socket; interior refers to residing within the socket. The exterior end 155 of the strap is releasably securable to a site on the exterior of the prosthetic socket 200, and the interior end of the strap is connectable to a distal end of a prosthetic socket liner 160 disposed within the socket. Strap 150 may be more generally referred to as a lanyard, and although typically having a flattened profile, it may also have a round profile or be of any suitable shape. Strap 150 embodiments may vary in thickness and width, according to particular of the prosthetic socket and patient needs.

In some embodiments, the interior end 152 of strap 150 is configured to support a bolt 153 (or any suitable connecting element) that is directed vertically toward the distal end 162 of the socket liner 160 and attachable thereto at a distal attachment site 163; vertical in this context refers to a being in alignment with the central longitudinal axis of the prosthetic socket. According, the bolt is disposed at an angle deviating from the longitudinal axis of the strap within a range of about 15 to about 40 degrees such that the angle of deviation is about the same as the angle by which the strap channel deviates from the horizontal. This configuration of the interior end 152 of strap 150 with a bolt 153 attached thereto is thus asymmetric, and is particularly arranged such that when strap 150 is tightened (by a pull from the exterior) to exert a pull that is directed substantially vertically downward in spite of the angled approach of the strap.

The responsiveness of prosthetic liner 160 being pulled by an angled strap is substantially better than it would be were the strap being pulled from a horizontal approach, and the translational efficiency of strap pull force into a proximally directed force is similarly better than it would be were the strap being pulled from a horizontal approach. Thus, by at least two structural factors: (1) the angled approach of the strap, and (2) the angled arrangement of the bolt on the interior end of the strap, the disclosed arrangement is an improvement over a strap channel having a horizontal approach to connecting to the distal end of a prosthetic liner.

In some embodiments, distal disk 104 of the prosthetic socket suspension mechanism includes a resilient shock-absorbing portion 122, such as a variable elastic modulus cushion. This padded structure precludes the socket liner from being drawn into the base of prosthetic socket 200 and encountering a hard surface against which it bottoms out. Once bottomed-out, no further force can be applied, and the arrangement as a whole has no subtle or graded adjustability. The padded encounter of the liner against the distal disk allows for a steady downward pull to be maintained, and translated into shock absorbing comfortable experience for the prosthetic socket user without sacrificing any of the benefit of the secure attachment between liner 160 and socket 200.

Embodiments of the provided technology include a prosthetic socket suspension mechanism that has a distal base for a prosthetic socket having a strap channel with an exterior opening into a central well of the base, the strap channel forming an angle inclined from the exterior opening toward the central well, the angle being within a range of about 10 to about 40 degrees from horizontal. The distal base has a distal disk and a proximal disk, the central well being formed within both the distal and proximal disks. The distal and proximal disks are rotatable and lockable with respect to each other within a range of about 10 degrees to about 25 degrees.

In some embodiments, the prosthetic suspension mechanism further includes a strap disposed through the strap channel, the strap having an exterior end, an interior end, and a longitudinal axis, wherein the exterior end of the strap is releasably securable to a site on the exterior of the prosthetic socket, and wherein the interior end of the strap is connectable to a distal end of a prosthetic socket liner disposed within the socket.

In particular embodiments, the interior end of the strap is configured to support a connector directed vertically toward the distal end of the socket liner, the bolt being disposed at an angle deviating from the longitudinal axis of the strap within a range of about 15 to about 40 degrees such that the angle of deviation is about the same as the angle by which the strap channel deviates from the horizontal.

Further, in some embodiments, the distal disk of the prosthetic socket suspension mechanism includes a resilient shock-absorbing portion. Some of these shock-absorbing embodiments include a cushion that includes a thermoplastic elastomer composition formed by a 3D printing process.

Cushion 122 may also be described as an article that has regions that vary in durometer. The terms “elastic” and “durometer” describe similar properties of material; an article that has a high modulus of elasticity (Young's modulus “E”) has a high resistance to being non-permanently deformed. An article having a high durometer has a high degree of hardness; thus, generally, an article having a high modulus of elasticity also has a high durometer. In the present application, variable modulus of elasticity will be used when describing aspects of embodiments of cushion 122. Modulus of elasticity is material property or attribute that includes directionality. Whereas durometer generally refers to hardness of an object as a whole, an article may have moduli of elasticity that vary according to the direction of the deforming force applied to it. Accordingly, this property of directionality can be controlled through the design delivered by way of 3D printing, and such controllable directionality of varying elasticity (or hardness) can be exploited for therapeutic advantage or specificity.

Variable elastic modulus cushions have widespread applicability to devices that interface with the body, particularly at sites where a graded quality of cushioning is appropriate or preferable, or when the site being cushioned is one where the interfacing body part is sensitive or vulnerable, and/or where the site is one through which the transfer of forces relates to functionality of the cushioned article. It is a challenge for a cushion to elastically engage at low levels of impinging force and not “bottom out” at a high level of impinging force. If a cushion bottoms out, then it actually no longer is acting as cushion. And, on the other hand, if a cushion is too hard (albeit not bottoming out), it doesn't really fulfill its mission or potential as a cushion. Thus, embodiments of a variable elastic modulus cushion 122, as provided herein, typically have a low elastic modulus on a surface that engages the body, and the elastic modulus increases with increasing depth within the cushion. The effect of such embodiments is that they provide a graded range of elasticity or hardness, and one that can be controlled by the design of the pattern and density of the 3D printed thermoplastic fill.

The embodiments of a variable elastic modulus cushion 122 provided herein represent but one of many examples of the utility of such cushions or pressure distribution pads that include internal variation in their modulus of elasticity or durometer, such variation being controllable and customizable. As a non-comprehensive listing, other examples include sites in prosthetic devices other than the distal cup of a prosthetic socket, as well as orthotic devices, exoskeletal devices, gripping elements in tools or utensils, and sites of bodily contact on walkers, canes, wheelchairs, stationary chairs, and beds. These examples of cushions of pads typically occur at sites in devices that patients engage with particular frequency or particular force, thereby creating a risk of irritation or injury of the engaging portion of the body.

“Customizable” or “customized”, as used herein, generally relates to fabrication of an article that replicates a contour (or a complement to a contour) of a body portion of an individual patient. The ability of a technology such as 3D printing to perform such replication requires the particular capabilities of creating a form with high resolution, and with high fidelity to the intended form. In addition to applying these 3D printing capabilities to the create replicative custom forms, they may also be directed to creating standardized (not individualized) or predetermined forms and patterns, ranging from simple to complex. These standardized forms and patterns can be generated in large arrays as models, individual actual devices from which are 3D printed only as needed.

This approach, in some instances, may allow fitting of fabricated articles to an individual patient with a specificity that rivals that of more strictly individualized custom fabrication. Forms and patterns typically can be described in dimensional or geometric terms (length, width, volume, etc.) but may also include intra-volume variation with regard to thermoplastic elastomer fill density among volume subsets of the article's total volume. This latter capability manifests as an article having internal regional variations in durometer and elasticity within the dimensional metrics.

Typically, variable elastic modulus cushion embodiments 122 are formed by 3D printing process. Accordingly, such cushions can also assume custom-shaping aspects or surfaces that conform to a body portion. Aspects of custom shaping prosthetic socket components by way of mass customization methods of manufacturing are described in U.S. patent application Ser. No. 14/572,571, as filed on Dec. 16, 2014, and U.S. Provisional Patent Application No. 62/007,742, as filed on Jun. 4, 2014. 3D printing methods offer a number of advantages over conventional approaches to fabricating a variable elastic modulus cushion or similar product that might approximate these structures, included among the advantages is the absence of a need for molds, which themselves consume resources.

Embodiments of a variable elastic modulus cushion 122 or related articles with portions that interface between a device and a portion of the body may be formed from any suitable 3D printing medium that yields a resilient body-friendly article. One broad class of suitable media includes thermoplastic elastomers. In one example, variable elastic modulus cushions may be printed with NinjaFlex® flexible filaments (thermoplastic polyurethanes) from Fenner Drives, Inc. (Manheim, Pa. 17545).

In the examples of a variable elastic modulus cushion embodiments 122 shown in FIG. 6A, the elasticity of cushion 122A is substantially uniform throughout the interior of the cushion (as indicated by a uniform interior structure). However, FIGS. 7A and 7B show interiors that have a relatively low density of material in the upper portion of the cushion and a relatively high density of material in the lower portion of the cushion. Thus the article will be easily deformed and provide relatively little resistance as the distal end of a wearer's residual limb initially impinges on the cushion. However, as pressure from the residual limb increases, so too does the elastic resistance to pressure. The relative proportion of 3D-printed fill and 3D-non-printed void can be controlled to the extent that the vertical profile of modulus of elasticity disallows the bottoming-out of the residual limb of a wearer against the distal surface of the cushion.

There is a difference, however, between the cross sectional profiles of cushions 122B (FIG. 7A) and 123C (FIG. 7B). Cushion 122B includes spheroidal void spaces that will deliver a broadly uniform elasticity with respect to directionality; the modulus of elasticity will be substantially the same in any direction from any reference point within the cushion. However, cushion 122C has 3D-printed walls or filaments oriented in particular directions; some structure is aligned vertically, some structure is aligned diagonally. Depending on the point of reference within cushion 122B, thus, elastic resistance to deformation will have a bias along the vectors defined by the walls and filaments. In a similar way, the relatively density of 3D-printed structure can vary circumferentially, such that, for example, an anterior portion of the cushion may have a relatively high modulus of elasticity while the circumferentially opposite posterior portion has a relatively low modulus of elasticity. Further still, the relative proportion of 3D-printed fill and void could vary regionally within the volume of a cushion, such that regular or irregular regions could vary from their immediately neighboring areas. Those of skill in the art can envision many variations of this theme, all of which are included in the scope of the present invention.

Embodiments of cushions that have variable or controllable, or directionally controllable variable elasticity may be applicable to many devices or applications, all highly customizable. In one example, the distal end of a residual limb can be replicated in a digital model, and that model applied to creating a control file for printing a cushion, such as variably elastic cushion 122, to be positioned in the distal well of a prosthetic socket. In this example, the model of the distal end of the residual limb, having a convex shape, is rendered into a complementary concave profile that prints a relatively forgiving, low fill density portion of thermoplastic elastomer within a surrounding bed of higher fill density.

A variably elastic modulus cushion 122 is but one example; FIGS. 15-16 show other embodiments. At least two aspects of the cushioning provided by the technology can be noted. In one aspect, 3D printing provides a complementary or body portion-conforming article that can be deliberately modified to accommodate considerations other than strict conformability, such as biomechanical considerations or personal preferences of the wearer. This capability stands as having therapeutic advantages even absent the use of the properties of controllable variable elasticity. The ability to deliver these structural features by digitally controlled 3D printing makes cushion 122 embodiments highly customizable for individual patients.

In the second aspect, however, variable elasticity, and the directionality that can be imparted to a cushion, and add further therapeutic capability to considerations strictly related to conformability in a surface engagement sense. For example, areas of the body that have been injured or are vulnerable to injury can be specifically protected. In some cases, this may involve creating inelastic, harder, and consequently protective portions adjacent to such vulnerable areas. Another therapeutic strategy is to create a more inelastic protective ring around a vulnerable site that directs impinging force away from the vulnerable site. A still further therapeutic strategy is less related to the vulnerability of an anatomical site due to injury or lesion, and more directed to protecting a site that is healthy but is subject to injury from the environment, or from particulars of a hazardous occupation. An example of this type of strategy underlies conventional baseball batting helmets, which are particularly protected on the side of the batter's head that is facing the pitcher.

3D printing of cushion 122 or similar articles can generally and, at least theoretically occur with the article being incrementally built out in any orientation on the 3D printing bed. For example, cushion 122 is typically printed out so that it is delivered in a horizontal, pancake-on-a-plate orientation. Practical considerations play a role in the appropriateness of orientation, however. For example, the horizontal orientation for printing a cushion 122 embodiment is efficient in terms of required printing time. An alternative orientation could require more time spent with unproductive movement of the printing nozzle. Other practical considerations regarding orientation relates to internal structure and the potential of distortion or collapse of delicate structure before it hardens after being laid down as a heated liquid.

Any one or more features of any embodiment of the inventions disclosed herein (device or method) can be combined with any one or more other features of any other embodiment of the inventions, without departing from the scope of the inventions. It should also be understood that the invention is not limited to the embodiments that are described or depicted herein for purposes of exemplification, but is to be defined only by a fair reading of claims appended to the patent application, including the full range of equivalency to which each element thereof is entitled.

1. A distal cup for a prosthetic socket for use with a residual limb of a patient, the distal cup comprising:
a proximal disk;
a distal disk; and
a cushion incorporated into the proximal disk and custom manufactured specifically for the patient, the cushion comprising:
a total cushion volume;
a volume of solid thermoplastic elastomer within the total cushion volume;
a void volume within the total cushion volume;
a first subset volume comprising a first relative thermoplastic elastomer fill volume defined by a ratio of a first solid thermoplastic elastomer volume within the first subset volume to a first total volume of the first subset volume; and
a second subset volume comprising a second relative thermoplastic elastomer fill volume defined by a ratio of a second solid thermoplastic elastomer volume within the second subset volume to a second total volume of the second subset volume,
wherein the first and second relative thermoplastic elastomer fill volumes are different from one another, and
wherein the first and second relative thermoplastic elastomer fill volumes are selected based at least in part on at least one physical characteristic of the patient.

2. The distal cup of claim 1, wherein the proximal disk and the distal disk each comprises a central well configured to allow passage of a strap lanyard of the prosthetic socket therethrough.

3. The distal cup of claim 1, wherein the proximal disk and the cushion comprise one, monolithic structure.

4. The distal cup of claim 1, wherein the cushion comprises a 3D printed cushion, and wherein the first and second relative thermoplastic elastomer fill volumes are formed via a 3D printing process.

5. The distal cup of claim 1, wherein a durometer of the cushion is selected for the patient by selecting the first and second relative thermoplastic elastomer fill volumes.

6. The distal cup of claim 1, wherein a modulus of elasticity of the cushion is selected for the patient by selecting the first and second relative thermoplastic elastomer fill volumes.

7. The distal cup of claim 1, further comprising a third subset volume within the total cushion volume, the third subset volume comprising a third relative thermoplastic elastomer fill volume defined by a ratio of a third solid thermoplastic elastomer volume within the third subset volume to a third total volume of the third subset volume, wherein the first, second and third relative thermoplastic elastomer fill volumes are different from one another.

8. The distal cup of claim 7, further comprising a fourth subset volume within the total cushion volume, the fourth subset volume comprising a fourth relative thermoplastic elastomer fill volume defined by a fourth ratio of a fourth solid thermoplastic elastomer volume within the fourth subset volume to a total volume of the fourth subset volume, wherein the first, second, third, and fourth relative thermoplastic elastomer fill volumes are different from one another.

9. The distal cup of claim 7, wherein the cushion comprises a spatial center and x, y, and z axes, and wherein the first, second and third relative thermoplastic elastomer fill volumes together form a fill volume gradient along one or more of the x, y, and z axes.

10. The distal cup of claim 7, wherein the cushion comprises a spatial center surrounded by ring-shaped layers emanating outward from the spatial center toward an external boundary, and wherein the first, second and third relative thermoplastic elastomer fill volumes together form a fill volume gradient on a line from the center through the ring-shaped layers.

11. The distal cup of claim 1, wherein the thermoplastic elastomer composition of the cushion comprises multiple structures selected from the group consisting of spheroidal structures, ovoid structures, walled structures, filamentous structures and irregularly-shaped structures.

12. The distal cup of claim 1, wherein the solid thermoplastic elastomer of the cushion comprises one or more structures, each structure comprising one or more vector orientations, and wherein a modulus of elasticity of the cushion varies through the cushion in accordance with the one or more vector orientations of the structures.

13. The distal cup of claim 1, wherein the 3D printing process is controlled by a control file comprising data providing an external boundary profile of the cushion and a map of the internal volume of the cushion.

14. The distal cup of claim 13, wherein the at least one physical characteristic of the patient comprises a digital profile of the patient's residual limb, and wherein the external boundary profile is derived, at least in part, from the digital profile of the patient's residual limb.

15. The distal cup of claim 13, wherein the control file comprises data that prescribe a range of predetermined sizing options for the cushion.

16. The distal cup of claim 13, wherein the control file comprises data that prescribe a variety of predetermined shape options for the cushion.

17. The distal cup of claim 1, wherein the at least one physical characteristic of the patient is selected from the group consisting of a weight of the patient, a height of the patient, a dimension of the residual limb, a digital profile of the residual limb, a status of an opposite limb of the patient, and a health condition of the patient.

18. A method of making a distal cup of a prosthetic socket for a residual limb of a patient, the method comprising:
fabricating a distal disk of the distal cup;
fabricating a proximal disk of the distal cup, wherein the proximal disk comprises a cushion custom manufactured specifically for the patient, the cushion comprising:
a total cushion volume;
a volume of solid thermoplastic elastomer within the total cushion volume;
a void volume within the total cushion volume;
a first subset volume comprising a first relative thermoplastic elastomer fill volume defined by a ratio of a first solid thermoplastic elastomer volume within the first subset volume to a first total volume of the first subset volume; and
a second subset volume comprising a second relative thermoplastic elastomer fill volume defined by a ratio of a second solid thermoplastic elastomer volume within the second subset volume to a second total volume of the second subset volume,
wherein the first and second relative thermoplastic elastomer fill volumes are different from one another, and
wherein the first and second relative thermoplastic elastomer fill volumes are selected based at least in part on at least one physical characteristic of the patient; and
coupling the distal disk to the proximal disk to form the distal cup.

19. The method of claim 18, wherein the cushion is fabricated via a 3D printing method, comprising:
providing instructions to a 3D printer via a control file, the control file comprising:
a map of an external boundary of the cushion;
a map of a total internal volume of the cushion, including the first and second relative thermoplastic elastomer fill volumes; and
printing the cushion with the 3D printer in accordance with the instructions.

20. The method of claim 19, wherein the instructions specify that the solid thermoplastic elastomer comprises multiple structures selected from the group consisting of spheroidal structures, ovoid structures, walled structures, filamentous structures, and irregularly-shaped structures.

21. The method of claim 19, wherein the instructions specify that the solid thermoplastic composition volume comprises one or more structures, each structure comprising one or more strength vector orientations, and wherein a modulus of elasticity of the cushion varies through the cushion in accordance with the one or more strength vector orientations of the structures.

22. The method of claim 19, wherein the instructions specify that the cushion comprise a spatial center and x, y, and z axes, and wherein the first and second relative thermoplastic elastomer fill volumes together form a relative fill volume gradient along one or more of the x, y, and z axes.

23. The method of claim 19, wherein the instructions specify the cushion comprise a spatial center surrounded by ring-shaped layers emanating outward from the spatial center toward an external boundary, and wherein the first and second relative thermoplastic elastomer fill volumes together form a relative fill volume gradient on a line from the center through the ring-shaped layers.

24. The method of claim 19, wherein the instructions specify that the first relative thermoplastic elastomer fill volume comprises an insular volume and the second relative thermoplastic elastomer fill volume comprises a region surrounding the insular volume.
